(19) United States
(12) Patent Application Publication
(10) Pub. No.: US 2024/0117293 A1
(43) Pub. Date: Apr. 11, 2024

(54) Monitoring System with Fiber Ripple Detection and Method

(71) Applicant: NIRRIN TECHNOLOGIES, INC., Billerica, MA (US)

(72) Inventors: Bryan A. Hassell, Cambridge, MA (US); Gregory A. Phelps, Cambridge, MA (US); David P. Marchessault, Hopkinton, MA (US)

(21) Appl. No.: 18/483,882

(22) Filed: Oct. 10, 2023

Related U.S. Application Data
(60) Provisional application No. 63/414,296, filed on Oct. 7, 2022.

(51) Int. Cl. C12M 1/00; G01N 21/27

(52) U.S. Cl. CPC C12M 41/06; G01N 21/274; G01N 2201/06113; G01N 2201/0683

(57) ABSTRACT

Devices for monitoring a bioreactor are configured for in-situ or ex-situ analysis. Noise from polarization dependent loss is addressed with a polarizer and reference photodetector for detecting a swept wavelength signal from a tunable laser spectrometer after transmission on a optical fiber but before a sample detection region.

### RELATED APPLICATIONS

This application claims the benefit under 35 U.S.C. § 119(e) of U.S. Provisional Application No. 63/414,296, filed on Oct. 7, 2022, which is incorporated herein by reference in its entirety.

### BACKGROUND OF THE INVENTION

Many processes in the chemical, biochemical, pharmaceutical, food, beverage and in other industries require some type of monitoring.

Sensors have been developed and are available to measure pH, dissolved oxygen (DO), temperature or pressure in-situ and in real-time. Common techniques for detecting chemical constituents include high performance liquid chromatography (HPLC), gas chromatography-mass spectroscopy (GCMS), or enzyme- and reagent-based electrochemical methods.

While considered accurate, many existing approaches for monitoring substances in a reactor are conducted off-line, tend to be destructive with respect to the sample, often require expensive consumables and/or take a long time to complete. In many cases, the equipment needed to perform these analyses is expensive, requires involved calibrations, and trained operators. Procedures may be time- and labor-intensive, often mitigated by decreasing the sampling frequency of a given process, thus reducing the data points. Often, samples are run in batches, after the process has been completed, yielding little or no feedback for adjusting conditions on an ongoing basis. Drawbacks such as these can persist even with automated sampling operations.

Various optical spectroscopy approaches are available to assess components, also referred to as analytes, in a sample. Among these, probably the most common is absorption spectroscopy. Incident light excites electrons of the analyte from a low energy ground state into a high energy, excited state, and the energy can be absorbed by both non-bonding n-electrons and π-electrons within a molecular orbital. Absorption spectroscopy can be performed in the ultraviolet, visible, and/or infrared region, with analytes of varying material phases and composition being interrogated by specific wavelengths or wavelength bands of light. The resulting transmitted light is then used to resolve the absorbed spectra, to determine the analyte's or sample's composition, temperature, pH and/or other intrinsic properties for applications ranging from medical diagnostics, pharmaceutical development, food and beverage quality control, to list a few.

One particular area of interest is the on-line and off-line monitoring of bioreactors that are widely used in various fields, including biotechnology and chemical engineering. Bioreactors are essentially vessels or systems designed to support the growth of microorganisms or cells, allowing for controlled biological processes. There are several common examples. In fermentation, they are used to produce a wide range of products, including pharmaceuticals, biofuels, and food products. Microorganisms such as bacteria, yeast, or fungi are cultivated in bioreactors to produce desired substances like antibiotics, ethanol, or enzymes. In biopharmaceutical manufacturing, bioreactors are used to culture mammalian cells for the production of therapeutic proteins, monoclonal antibodies, and vaccines. These systems must maintain strict control over environmental factors like temperature, pH, and oxygen levels to ensure the quality and yield of the final product. For tissue engineering, bioreactors provide a controlled environment for growing and maturing artificial tissues and organs. This involves culturing cells on scaffolds within bioreactors to create functional tissues. Other areas of use include algal cultivation, waste water treatment, bioremediation, research and development, food and beverage industry, and pharmaceutical screening, to list a few examples.

U.S. Pat. Pub. No. 2021/0088433 by Hassell et al., issued as U.S. Pat. No. 11,499,903B2 on Nov. 15, 2022, both being incorporated herein by this reference in their entirety, describes robust, hands-free, non-destructive, real-time techniques for identifying and/or quantifying constituents in a given process. Typically, the process is conducted in a vessel, e.g., a bioreactor. The contents of the bioreactor can change as the process unfolds and these changes are tracked using near infrared (NIR) spectrometry. Substances present in the bioreactor are identified using an in-situ probe that is inserted and/or maintained in a bioreactor. Thus, the analysis can be conducted in real time, in a nondestructive manner.

### SUMMARY OF THE INVENTION

Tunable laser spectrometers will typically have a wavelength reference detector and a power reference detector. The wavelength reference allows the device to track its wavelength sweep through the tunable laser's spectral scan band to ensure highly accurate wavelength resolution for resolving the absorption spectra. The power reference detector detects the instantaneous power during the sweep so that any changes in the laser's power as it sweeps can be compensated to accurately resolve the absorption spectra of the material of interest.

Nevertheless, for highest performance operation, all power variability must be fully compensated. And one source of power variability arises from the highly polarized nature of diode lasers used in many laser spectrometers. Small changes in the polarization in conjunction with polarization dependent loss (PDL) in the different components such as lenses, beam splitters, fibers and detectors will result in an untracked power variability that will degrade the accuracy of the measured absorption spectra.

In addition, sometimes polarization maintaining (PM) fiber, such as polarization maintaining single mode optical (PANDA) fiber, is used between the tunable laser spectrometer and probe and/or sample cell and/or sample holder that interfaces with the sample. This PM fiber ensures the polarization stability that is required to manage PDL. The use of polarization maintaining fiber does not entirely solve the problem, however. The phenomenon of fiber polarization beat or PANDA ripple also arises because there is usually some power in the non-preferred polarization and this optical mode will beat with the power in the preferred polarization mode, causing power fluctuations. More generally, when two waves with different linear polarization states propagate in the birefringent PM fiber, their phases will evolve differently. The difference in phase delay will be proportional to the fiber length.

In general, according to one aspect, the invention features a monitoring system comprising a tunable laser spectrometer for generating a swept wavelength signal for transmission on an optical fiber, a reference photodetector for detecting the swept wavelength signal from the tunable laser spectrometer after transmission on the optical fiber, and a sample photodetector for detecting the swept wavelength signal after transmission through a sample detection region.

In embodiments, a polarizer is provided for improving a polarization of the swept wavelength signal prior to the reference photodetector.

In addition, the optical fiber can include polarization maintaining fiber. One example is single mode polarization maintaining fiber.

In some embodiments, a sample interface includes an optical transmission port and optical detection port including at least one beveled surface, such as provided by an input waveguide rod and output waveguide rod.

In one example, the tunable laser spectrometer sweeps its wavelength in a spectral band including 2.3 micrometers and/or 6.5 micrometers.

It can be implemented as a probe that is inserted into a bioreactor or can include a sample interface for receiving drops of a sample extracted from a bioreactor.

Typically, a controller is provided monitors a response of the sample photodetector and the reference photodetector to resolve an absorption spectra of a sample in the sample detection region and preferably the controller compensates for noise associated with ripple from the optical fiber and/or temperatures detected by one or more thermistors associated the reference photodetector and the sample photodetector.

Generally, no optical fiber is provided after the reference detector to the sample photodetector. This way the reference detector can be used to compensate for any polarization sensitivity in the system.

In general, according to another aspect, the invention features a monitoring system comprising a tunable laser spectrometer for generating a swept wavelength signal, a reference photodetector for detecting the swept wavelength signal from the tunable laser spectrometer, a polarizer for improving a polarization of the swept wavelength signal prior to the reference photodetector, and a sample photodetector for detecting the swept wavelength signal after transmission through a sample detection region.

In general, according to another aspect, the invention features a probe for a bioreactor comprising ports defining a sample detection region in the bioreactor, a reference photodetector for detecting an optical signal received from a polarizer and prior to the sample detection region, and a sample photodetector for detecting the optical signal after transmission through the sample detection region.

In general, according to another aspect, the invention features method for on-line or off-line monitoring of a bioreactor. The method comprises generating a swept wavelength signal, transmitting the swept wavelength signal to a sample detection region for a sample of the bioreactor, polarizing the swept wavelength signal, detecting the swept wavelength signal after polarization and prior to transmission through the sample detection region, detecting the swept wavelength signal after transmission through the sample detection region, and resolving an absorption spectra of a sample in the sample detection region with reference to the swept wavelength signal before and after transmission through the sample detection region.

Techniques such as the ones described herein also improve the quality of the analysis. For example, embodiments described herein can provide improved or even maximum signal to noise ratios. This is accomplished by launching a light beam straight out of a fiber and/or a free space link, through a sample gap, with the transmitted light impinging onto a photodetector. By having the detector cables running up the length of the probe, rather than using a return fiber optic cable leading to a photodiode, often external to the reactor, approaches described herein can rely on the signal to noise ratio (SNR) of the electrical cables, which generally are superior to fiber optics.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a schematic view of a monitoring system including a tunable laser spectrometer and an in-situ probe, according to the invention;

FIG. 2 is cross sectional view showing head section of the probe;

FIG. 3 is cross sectional view showing tip section of the probe;

FIG. 4 is side perspective view of the cross-sectional view showing rods in the tip section of the probe; and

FIG. 5 is a side plan view showing another monitoring system according to the invention.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Many processes conducted in vessels such as bioreactors require or benefit from a stringent control of parameters such as pH, levels of oxygen, nutrients, metabolites, waste products and/or other species. In many of its aspects, the invention relates to a device and method for analyzing the contents of a bioreactor, often on an ongoing basis, off-line or on-line. Reactor components can be detected, at various time intervals and the data can be used to assess conditions and, if necessary, adjust or optimize process parameters. Examples of processes that can be monitored include cell growth protocols, biopharmaceutical manufacturing, tissue engineering, fermentations, algal cultivation, waste water treatment, bioremediation, research and development, food and beverage industry, and so forth.

In one implementation, techniques described herein are practiced with a bioreactor that houses or is a cell culture system for the three-dimensional assembly, growth and differentiation of cells and tissues. The bioreactor typically contains a combination of cells, culture media, nutrients, metabolites, enzymes, hormones, cytokines and so forth.

Analysis can employ a spectroscopy system for determining the spectral response of the components in a sample detection region in one or more of the following spectral regions: infrared (IR), including near-, mid- and/or far-infrared, visible, and/or ultraviolet (UV). Further, the spectroscopy system can measure different characteristics, such as absorption spectra, emission (including blackbody or fluorescence) spectra, elastic scattering and reflection spectra, impedance (e.g., index of refraction) spectra, and/or inelastic scattering (e.g., Raman and Compton scattering) spectra, of analytes in the bioreactor.

Specific embodiments described herein rely on spectroscopy in the ultraviolet and visible regions, but mostly in the infrared region extending from 700 nanometers (nm) to 1 millimeter (mm) in wavelength and specifically including the near infrared (0.75-1.4 μm, NIR), short-wavelength infrared (1.4-3 μm, SWIR), mid-wavelength infrared (3-8 μm, MWIR), long-wavelength infrared (8-15 μm, LWIR), and/or the far infrared (15-1000 μm, FIR) of the spectrum. Probing molecular overtone and combination vibrations, NIR-SWIR spectroscopy covers the region of from 750 nanometer (nm) to 2500 nm of the electromagnetic spectrum. In one embodiment the tunable laser sweeps its wavelength in a spectral band including 2.3 micrometers and more specifically sweeps through greater than 100 nanometers around 2.3 micrometers. In one example, the laser sweeps its wavelength over greater than 150 or 200 nanometers such as from about 2.2 to 2.4 micrometers. An overview of NIR spectroscopy can be found, for example, in an article by A.M.C. Davies in “An Introduction to Near Infrared (NIR) Spectroscopy”, impublications.com/content/introduction-near-infrared-nir-spectroscopy. See also, Cervera, A. E., Petersen, N., Lantz, A. E., Larsen, A. & Gernaey, K. V. “Application of near-infrared spectroscopy for monitoring and control of cell culture and fermentation,” Biotechnol. Prog. 25, 1561-1581 (2009); and Roggo Y, et al., “A review of near infrared spectroscopy and chemometrics in pharmaceutical technologies”, Journal of Pharmaceutical and Biomedical Analysis, Volume 44, Issue 3, 2007.

According to embodiments described herein, measurements are taken within (in-situ) the reactor, typically without a need to withdraw a sample into a sample or flow cell, for on-line monitoring or into an external (ex-situ) arrangement for taking an off-line reading.

Many implementations of the present invention employ one or more outer tubes of for example stainless steel which house one or more photodetectors, such as a photodiode, and one or more optical components such as free space transmission paths, optical fibers, other waveguide(s) and/or window(s) and/or focusing lens(es). These elements define a sample interface with an optical transmission port and an optical detection port defining a sample detection region. Being in contact with the fluid inside the bioreactor or extracted therefrom, the spacing between these windows can be the pathlength of the laser light through the fluid.

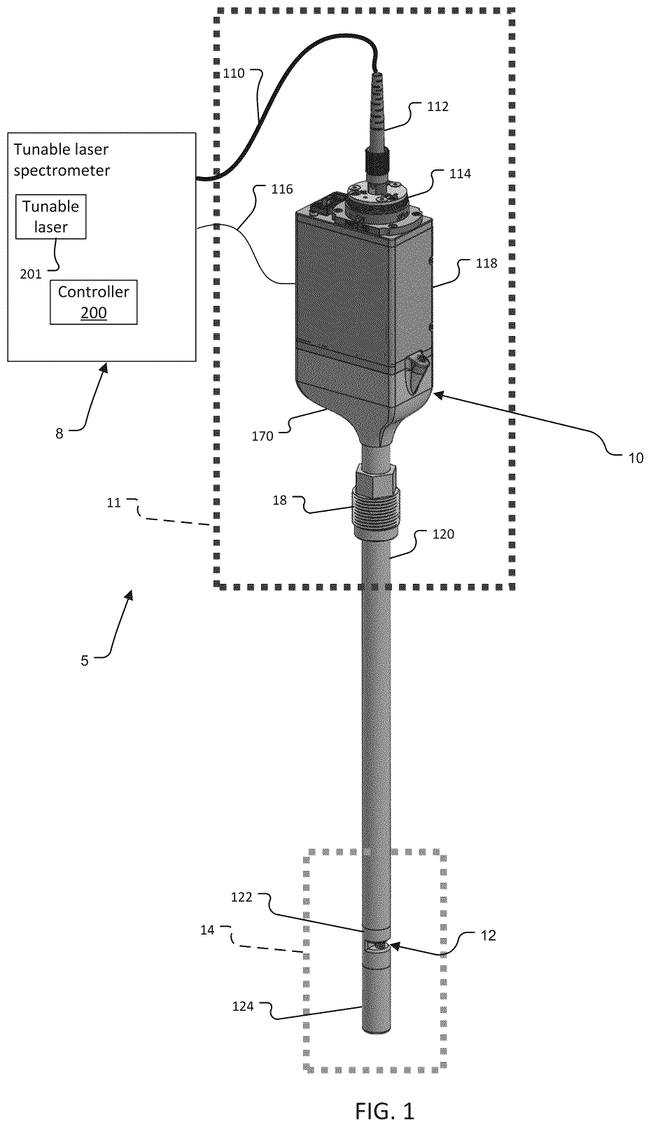

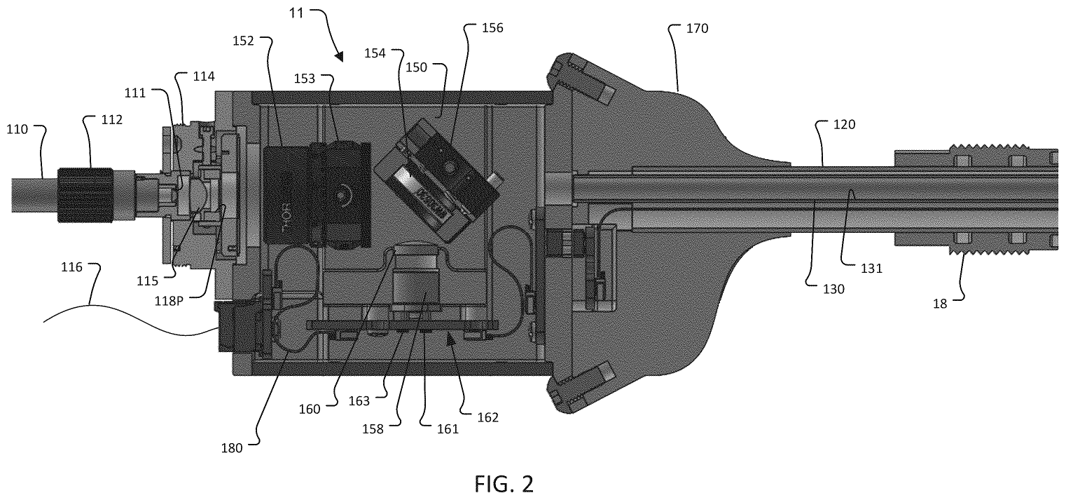

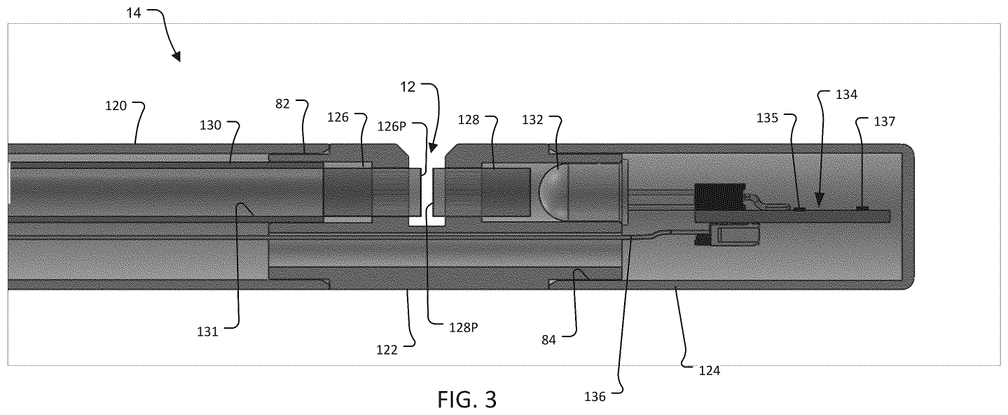

FIG. 1 shows monitoring system 5 comprising a tunable laser spectrometer 8 and the in-situ probe 10 providing a sample interface. This probe is configured for placing and/or maintaining the sample interface as a sample detection region 12 within a vessel such as bioreactor. FIG. 2 is a cross-sectional view of a head section 11 of the in-situ probe 10 and FIG. 3 is a longitudinal cross-sectional view of a tip section 14 of the in-situ probe 10.

The tunable laser spectrometer 8 is connected to the in-situ probe 10 via an optical fiber patch cable 110. It terminates with an optical fiber connector 112 which mates with an optical fiber receptacle 114 on a housing 118 of the head section 11.

A spectrometer electrical wiring harness 116 electrically connects the tunable laser spectrometer 8 with the head section 11.

In general, the head section 11 is characterized by the probe optical housing 118 that mates with an outer metal, e.g., 12 millimeter (mm) outer diameter (OD) stainless steel, probe tube 120 at its lower end.

The outer metal probe tube 120 terminates in a sample assembly 122, with a lower end tube 124 below the sample assembly in the tip section 14.

Since many bioreactor headplates are equipped with ports for receiving various fittings which can be screwed in, a fitting 18 is provided for making a seal on the headplate at the top a bioreactor. In more detail, fitting 18 can be a PG13.5 fitting having the standard thread typically used on bioreactor headplates.

FIG. 2 shows the internal components of the head section 11. The optical fiber patch cable 110 terminates with an APC-SM fiber connector 112 that connects to a compatible fiber receptacle or port 114. The port includes a collimating lens 115 for collimating the beam exiting from the fiber end face 111. The beam propagates through a port 118P in the probe optical housing 118.

In a current embodiment, the optical fiber patch cable 110 is PM and usually specifically PANDA fiber. This ensures that the polarization is stable between the tunable laser spectrometer 8 and the probe 10.

The collimated free-space beam of the light exiting the fiber is received by a polarizer 152 that is supported on a rotation mount 153. The polarizer 152 filters or removes the light in the orthogonal polarization. The rotation mount 153 secures the polarizer to a bench 150 of the head section 11. The rotation mount 153 allows for the rotation of the polarizer 152 in the plane that is orthogonal to the top surface of the bench 150 and orthogonal to the optical axis of the beam exiting from the fiber. During calibration, the rotation mount 153 is used for fine rotational adjustment of the polarizer 152 so that it is aligned to the preferred polarization axis of the PANDA fiber.

A partially reflecting sapphire window 154, such as a wedge window, is held on a pitch yaw mount 156 that secures the window 154 to the bench 150. The partially reflecting sapphire window 154 reflects a portion of the beam to a ripple reference photodetector 158, such as an In—GaAs detector. A focusing lens 160 couples the beam onto the active area of the ripple reference detector 158. The ripple detector is mounted to a head printed circuit board (PCB) 162, which includes a transimpedance amplifier 163 to amplify the electrical response of the ripple reference detector. A thermistor 161 is also preferably provided on the head printed circuit board 162 to allow for temperature compensation of the detector 158 and transimpedance amplifier 163. The response of the ripple reference photodetector is then transmitted through internal harness 180 and the spectrometer electrical wiring hardness 116 to the spectrometer 8.

The pitch yaw mount 156 allows the adjustment of the free space beam reflected and transmitted through the sapphire window 154 so that a portion of the beam will propagate down the center of the inner bore 131 of an inner tube 130 within the outer tube 120, while the other portion will strike the active area of the ripple reference detector 158.

With reference to FIG. 3, the sample detection region 12 is located in the tip section 14 of probe 10 and is positioned with an indentation formed in the sample assembly 122. The sample detection region 12 is defined by an optical transmission port 126P and an opposed optical detection port 128P. In the current example, the optical transmission port 126P is formed by a quartz or sapphire input waveguide rod 126 that is held in the body of the sample assembly 122; the optical detection port 128P is formed by a quartz or sapphire output waveguide rod 128 that also is held in the body of the sample assembly 122. The gap between the two rods and their ports defines the sample detection region 12 and, typically, the pathlength of the light traversing the sample.

The input waveguide rod 126 optically couples to free space path defined by the inner bore 131 of the inner tube 130. The output waveguide rod 128 optically couples to a sample photodetector 132. In the current example, the sample photodetector 132 is a dome lens TO-46 In—GaAs detector. The sample photodetector 132 electrically connects to a sample detector PCB 134 housed in the lower end tube 124. The detector PCB 134 includes its own transimpedance amplifier 135 for amplifying the detector response and transmitting that response through an internal wiring harness 136 to the head PCB 162 and then through the spectrometer electrical wiring hardness 116 to the spectrometer 8. A thermistor 137 is included on detector PCB 134 to detect the temperature including the temperature of the in the tip, allowing for temperature offsets.

The sample assembly 122 fits into the outer metal probe tube 120 at its an upper end and fits into the lower end tube 124 at its lower end. Specifically, an upper necked-down portion 82 is inserted into the lower end of outer metal probe tube 120. The outer surface of the necked-down portion 82 is bonded to the inner surface of the lower end of the outer metal probe tube 120. Similarly, a lower necked-down portion 84 of the sample assembly 122 is inserted into the upper end of lower end tube 124.

The dimensions of tip section 14 and the outer metal probe tube 120 can be selected according to the size of the reactor. In many situations, the longitudinal distance between the fitting 18 and the optical detection region 12 of the tip section 14 is configured to expose detection area 12 to the reactor medium being monitored and specifically a portion of that medium that is representative of all of the medium in the bioreactor, rather than possibly unmixed medium along a wall of the reactor or near the surface of the liquid in the reactor. In one illustrative example, the distance between the fitting 18 and the optical detection region 12 is at least 1 centimeter (cm), e.g., at least 2, 3, 4, 5, 10, 15, 20, 25, 30, 35, 40, 45, 50, 55, 60, 65, 70, 75, 80, 85, 90, 95 or 100 cm. Tip section 14 can be smaller, for miniaturized reactor designs, for instance, or larger, for some industrial scale applications.

In general, during operation, the swept wavelength light from the tunable laser 201 is coupled into the optical fiber patch cable 110 to the probe optical housing 118. In one embodiment the tunable laser includes a gallium antimonide (GaSb) gain chip and sweeps its wavelength in a spectral band including 2.3 micrometers and sweeps through greater than 100 nanometers. In one example, the laser sweeps its wavelength from about 2.2 to 2.4 micrometers.

In another example, the tunable laser 201 operates in the MIR and includes a quantum cascade gain chip. There the tunable laser sweeps through a spectral band including 6.500 microemeters and preferably sweeps through greater than 500 nanometers or even greater than 1000 nanometers. One specific example sweeps through a range of about 5.5 micrometers to 7.5 micrometers.

The PANDA fiber optical fiber patch cable 110 acts to reject any higher order spatial modes from the laser and minimize ripple. But the fiber is lossy, so a short length is desired. Generally, the PANDA fiber optical fiber patch cable 110 between 0.5 meters and 3-4 meters in length. The polarizer 152 further removes any modes and random polarization fluctuations and addresses polarization dependent loss in the optical components by enforcing a single polarization. The ripple reference detector 158 further improves operation by addressing random power attenuation arising from the PANDA fiber optical fiber patch cable 110.

Light is polarized by the polarizer 152, removing the risk of polarization dependent loss in the optical components. And a portion of the polarized light is detected by the ripple reference photodetector 158. This ripple reference signal is transmitted back to the spectrometer 8 via the spectrometer electrical wiring harness 116.

The remaining light travels (from partially reflecting sapphire window 154) through the free space path defined by the inner bore 131 of the inner tube 130. The light is coupled into input waveguide rod 126 exiting out via optical transmission port 126P and then propagates through the sample detection region 12. The light leaves the sample detection region 12 through the optical detection port 128P and propagates through the output waveguide rod 128; it is then detected by the sample photodetector 132 after being modulated by the bioreactor medium in the region 12, which will preferentially absorb some wavelengths relative to others.

A controller 200 of the spectrometer 8 monitors the response of the sample photodetector 132 as well as the ripple reference photodetector 158. Thus, the controller can resolve the absorption spectra of the sample by monitoring the spectral scanning of the tunable laser 201 over its scan band relative to the time-response of the sample photodetector 132. Any noise associated with ripple or other sources from the optical fiber is compensated by the response from the ripple reference photodetector. Generally, the tunable laser or tunable laser system sweeps its narrow band emission over some region of the electromagnetic spectrum such as the NIR and/or SWIR regions, or portions thereof.

The controller 200 uses the temperatures detected by the thermistor 161 on the head PCB 162 and the thermistor 137 on the detector PCB 134 to compensate for change in the response of the ripple reference photodetector 158 and the sample photodetector 132 and for changes in the gain of the transimpedance amplifiers on the head PCB 162 and detector PCB 134 arising from temperature changes.

The probe optical housing 118 of the head section 11 is designed to be detachable, from a lower section 170 that is connected to the outer tube 120 and inner tube 130. This allows the head section to be removed so that the remainder of the probe can be autoclaved.

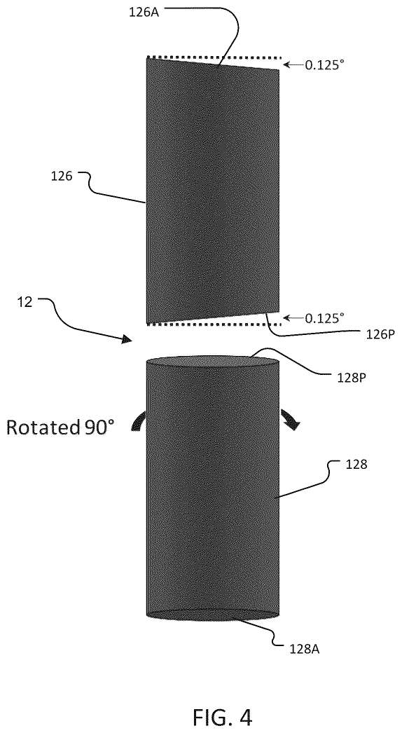

FIG. 4 illustrates some design details associated with the input waveguide rod 126 and the output waveguide rod 128.

Etalons can be formed between parallel reflecting surfaces. Surfaces will reflect due to the refraction index mismatch between air of the free space path of the beam through the inner tube 130 and the bulk material of the input waveguide rod 126. An index mismatch will also be typically present between the fluid in the sample region 12 and the input waveguide rod 126 and the output waveguide rod 128. The beam also propagates through free space between the output waveguide rod 128 and detector 132. And even if surfaces are antireflection coated, residual reflectivity will still be present.

In a preferred embodiment bevels are placed on ports 126A, 126P, 128P and 128A of the input waveguide rod 126 and the output waveguide rod 128. Typically, the bevels are a few tenths of a degree. 0.125 degrees is shown. Also, preferably the bevels of the input waveguide rod 126 and the output waveguide rod 128 are rotated 90 degrees relative to each other. In the example, the bevels of ports 126A, 126P of the input waveguide rod 126 are beveled left to right in the plane of the figure. The bevels of ports 128A, 128P of the output waveguide rod 128 are beveled fore and aft in the plane of the figure.

There are other systems that require polarization control for peak performance.

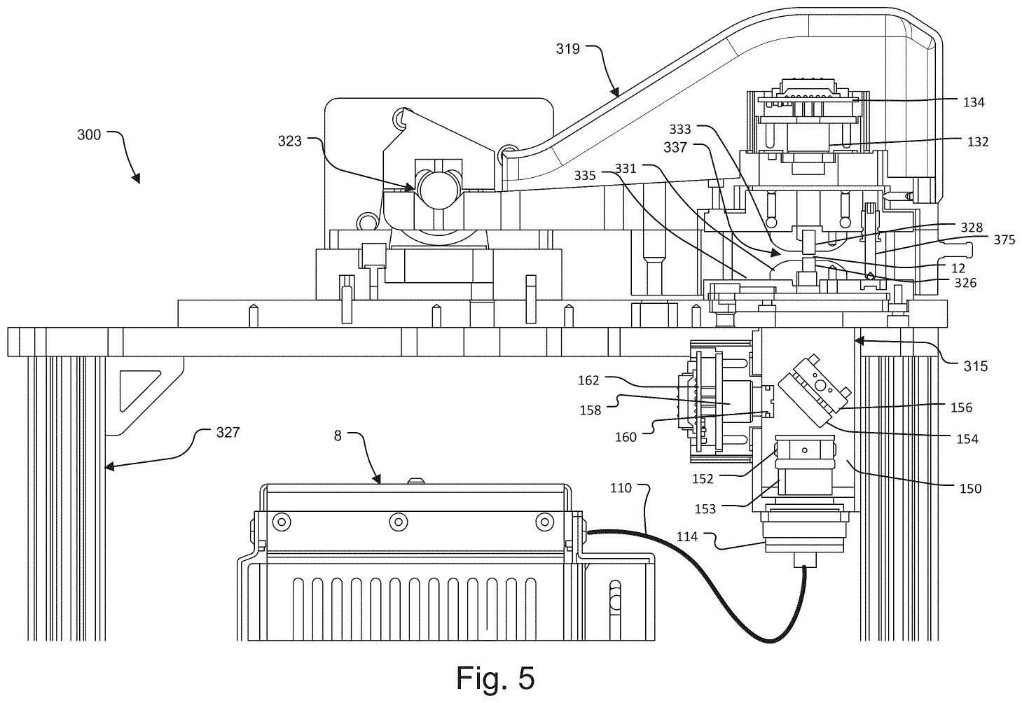

FIG. 5 shows another monitoring system 300 having a clamshell configuration, which is often used for off-line monitoring, but on-line monitoring is possible with an added flow cell. Details are described, for example, in U.S. Provisional Patent Application Numbers: 63/423,856, filed on Nov. 9, 2022 (under Attorney Docket No. 0376.0029PR1); 63/469,502, filed on May 29, 2023 (under Attorney Docket No. 0376.00029PR2); and 63/512,996, filed on Jul. 11, 2023 (under Attorney Docket No. 0376.0031PR1, the entire teachings of all three applications being incorporated herein by this reference.

A first section 315 is fixed and secured onto a base or support 327 and, in the current embodiment, represents the lower or bottom portion of a clamshell apparatus. In this configuration, the first section 315 hangs from support 327 and does not move or flip. Rather, the opening and closing movement is performed by the second section, section 319. In the current embodiment, second section 319 represents the upper, top or lid-like section. Other arrangements or orientations of the two sections are possible.

Top section 319, supported on support 327, provides a clam-like or “flip” arrangement, for opening and closing the apparatus. Various mechanisms can be employed to move the upper section between an open and closed configuration. For example, connection 323 can be or can include one or more of the following elements: jaws, hinges, soft close arrangements, rods, levers, or others, as known in the art. Lowering or lifting the top section 319 relative to bottom section 315 is performed by a knob, rod and soft close feature. Some approaches can provide a lip for lifting or lowering section 319, with the connection including a hinge mechanism, for example. In general, connection 323 can be constructed using elements and techniques known in the art.

In some implementations, connection 323 joins the two sections directly. For instance, it can be supported onto the fixed first section and can include a mechanism that allows attachment of the second section and its movement between an open and a closed configuration. Other implementations rely on an additional component for supporting connection 323 or components thereof. For instance, support 327 can be used to position top section 319 onto a structure, to ensure precision mating of the top and bottom sections when in the closed configuration.

During operation, section 319 can be opened to expose the sample interface 337, to receive a sample, in the form of a drop, for instance, then closed, much like a lid, for the sample analysis.

Light from the tunable laser in the tunable laser spectrometer 8 is transmitted via fiber optics 110. Electrical signals obtained from the photodetector(s) employed can be collected and transmitted to the tunable laser spectrometer 8 via cables, using one or more wire harnesses, for example. The tunable laser, along with the controller, are typically part of the tunable laser spectrometer 8.

Following a light beam generated by tunable laser in the spectrometer 8 and transmitted via an optical fiber 110, light enters section 315 at fiberport 114. In some implementations, fiber patch cable 110 includes PANDA fiber for transmitting the light from the tunable laser. Fiberport 114 can be configured as a collimator for light that exists the optical fiber patch cable and is directed to polarizer 152 (for filtering and removing the light in the orthogonal polarization). A rotational mount 153 allows for the rotation of polarizer 162 in a plane that is orthogonal to bench 150 and orthogonal to the optical axis of the beam exiting from the fiber. In some implementations, mount 153 provides fine rotational adjustment of the polarizer 152 so that it can be aligned to the preferred polarization axis of the PANDA fiber 110.

A beam splitter includes a partially reflecting sapphire window 154, such as a wedge window, held on a pitch yaw mount 156 that secures the window 154 to the bench 150. The partially reflecting sapphire window 154 reflects a portion of the beam, referred to herein as a “reference” beam, to a ripple reference photodetector 158, such as an In—GaAs detector. A focusing lens 160 couples the beam onto the active area of the ripple reference detector 158. In addition to holding the window 154, the pitch yaw mount 156 allows the adjustments of the free space beam reflected and transmitted through the sapphire window 154 so that the beam will propagate to strike the active area of the ripple reference detector 158.

The ripple detector is mounted to a head printed circuit board (PCB) 162, which includes a transimpedance amplifier to amplify the electrical response of the ripple reference detector. A thermistor can be provided on the head printed circuit board to allow for temperature compensation of the detector 158 and transimpedance amplifier. The response of the ripple reference photodetector along with the response of the thermistor can then be transmitted as an electrical signal to tunable laser spectrometer 8 via electrical connection, in an electrical wiring harness arrangement (not shown), for example.

In addition to the reference beam, the beam splitter generates a second beam portion, referred to herein as an “input” or “interrogation” beam. This beam propagates from partially reflecting sapphire window 154 towards a sample interface 337 and in particular towards sample detection region 12, defined by an optical transmission port and an opposed optical detection port. In the current example, the optical transmission port is formed by a quartz or sapphire input rod 326 and the optical detection port is formed by a quartz or sapphire output rod 328. The rod waveguide arrangement described herein obviates the need for cuvettes, a component typical of existing instrumentation.

In some implementations, rods 326 and 328 are held in by rod holders 331 and 333, respectively. One or both rod holders can be heated under the control of the controller in the spectrometer 8.

Input rod 326 is disposed at the sample pedestal 335, while output rod 328 is part of section 319 of clamshell apparatus. The open position allows introducing a sample (or, for calibration purposes, a blank) onto rod 326 at sample pedestal 335. Moving section 319 to a closed position brings the two rods in alignment; the distance between their ports defines the pathway travelled by an incoming (interrogation) beam through the sample detection region 12. Precision ball screw 375, extending from one end located in section 319 to sample pedestal 335, can be used to offset the rods and set the pathway to a fixed pathlength.

The selected pathlength between the end of the rods 326, 328 defining the sample detection region 12 can have a value within a range of from about 0.010 millimeters (mm) to about 5 mm or, in some cases, to about 10 mm, such as, for example, within a range of from about 0.01 mm to about: 0.05 mm, 0.1 mm, 0.5 mm, 1 mm, 2 mm, 3 mm, 4 mm, 5 mm, 6 mm, 7 mm, 8 mm, 9 mm, 10 mm; or from about 0.05 mm to about: 0.1 mm, 0.5 mm, 1 mm, 2 mm, 3 mm, 4 mm, 5 mm, 6 mm, 7 mm, 8 mm, 9 mm, 10 mm; or from about 0.1 mm to about: 0.5 mm, 1 mm, 2 mm, 3 mm, 4 mm, 5 mm, 6 mm, 7 mm, 8 mm, 9 mm, 10 mm; or from about 0.5 mm to about: 1 mm, 2 mm, 3 mm, 4 mm, 5 mm, 6 mm, 7 mm, 8 mm, 9 mm, 10 mm; or from about 1 mm to about: 2 mm, 3 mm, 4 mm, 5 mm, 6 mm, 7 mm, 8 mm, 9 mm, 10 mm; or from about 2 mm to about: 3 mm, 4 mm, 5 mm, 6 mm, 7 mm, 8 mm, 9 mm, 10 mm; or from about 3 mm to about: 4 mm, 5 mm, 6 mm, 7 mm, 8 mm, 9 mm, 10 mm; or from about 4 mm to about: 5 mm, 6 mm, 7 mm, 8 mm, 9 mm, 10 mm; or from about 5 mm to about: 6 mm, 7 mm, 8 mm, 9 mm, 10 mm; or from about 6 mm to about: 7 mm, 8 mm, 9 mm, 10 mm; or from about 7 mm to about: 8 mm, 9 mm, 10 mm; or from about 8 mm to about: 9 mm, 10 mm; or from about 9 mm to about 10 mm.

One illustrative pathlength is about 5 mm. Another illustrative pathlength is 1 mm. For mid-range IR (MIR) systems, the illustrative pathlengths can be reduced, to about 100 microns, for instance.

In specific implementations, the rods 326, 328 have a diameter within a range of from about 2 to about 8 millimeter (mm), e.g., about 4 mm. This arrangement can hold a drop of about 15 microliter (mL) through surface tension, while allowing a beam of between 0.5 and 3 mm in diameter, preferably about 1 mm, to progress through the rods and the sample in the sample detection region 12. In many cases, both rods have the same diameter. In others, the rods have different diameters. In one example, the base window is larger to allow for is cleaning.

Output rod 328 optically couples to a sample photodetector 132, provided with lens. The electrical signal registered by the photodetector 132 is transmitted to tunable laser spectrometer 8 via electrical connection, in a wire harness arrangement (not shown). In one example, the photodetector is a dome lens TO-46 In—GaAs detector which can be electrically connected to a sample detector printed circuit board (PCB) 134. The detector PCB can include its own transimpedance amplifier for amplifying the detector response and transmitting that response to tunable laser spectrometer 8. A thermistor can be included on the detector PCB to detect the temperature allowing temperature offsets for compensation by the controller.

During operation, the swept wavelength light from the tunable laser of spectrometer 8 is coupled into the optical fiber patch cable 110. The PANDA fiber optical fiber patch cable operates to reject modes, minimize ripple. Short fiber patch cable lengths reduce or minimize losses. Polarizer 154 further removes modes and random polarization fluctuations and addresses polarization dependent loss in the optical components. The ripple reference detector 158 is employed to further improve operations by addressing random power attenuation. Polarizing the light removes the risk of polarization dependent loss in the optical components by enforcing a stable polarization. A portion of the polarized light is detected by the ripple reference photodetector 158. This ripple reference signal is transmitted back to the tunable laser spectrometer 8 via a spectrometer electrical wiring harness, for example.

The remaining light travels toward and is coupled into input rod 326 exiting out the optical transmission port. It then propagates through the sample detection region 12. The light leaves the sample detection region 12 through the optical detection port and propagates through the output rod 328. It is detected by the sample photodetector 132 after being modulated by analytes in the sample detection region 12. The analytes will preferentially absorb some wavelengths relative to others.

In many cases, once it has entered the clamshell apparatus, the light beam is not confined to an optical fiber but travels (from one optical element to another) in free space.

The controller, which can be part of the tunable laser spectrometer 8 monitors the response of the sample photodetector 132 as well as the ripple reference photodetector 158. Thus, the controller can resolve the absorption spectra of the sample by monitoring the spectral scanning of the tunable laser over its scan band relative to the time-response of the sample photodetector 132. Any noise associated with ripple or other sources from the optical fiber is compensated by the response from the ripple reference photodetector 158. Generally, the tunable laser or tunable laser system sweeps its narrow band emission over some region of the electromagnetic spectrum such as the NIR and/or SWIR regions, or portions thereof.

The controller of the spectrometer uses the temperatures detected by the thermistor on the PCB associated with the sample detector 132 and the thermistor associated with the reference detector 158 to compensate for change in the response of the ripple reference photodetector 158 and the sample photodetector 132 and changes in the gain of the transimpedance amplifiers on the PCB s.

While this invention has been particularly shown and described with references to preferred embodiments thereof, it will be understood by those skilled in the art that various changes in form and details may be made therein without departing from the scope of the invention encompassed by the appended claims.

What is claimed is:

1. A monitoring system comprising:
a tunable laser spectrometer for generating a swept wavelength signal for transmission on an optical fiber;
a reference photodetector for detecting the swept wavelength signal from the tunable laser spectrometer after transmission on the optical fiber;
a sample photodetector for detecting the swept wavelength signal after transmission through a sample detection region.

2. The system of claim 1, further comprising a polarizer for improving a polarization of the swept wavelength signal prior to the reference photodetector.

3. The system of claim 1, wherein the optical fiber includes polarization maintaining fiber.

4. The system of claim 3, wherein the polarization maintaining fiber is single mode polarization maintaining fiber.

5. The system of claim 1, wherein the sample interface includes an optical transmission port and optical detection port including at least one beveled surface.

6. The system of claim 5, wherein the optical transmission port and the optical detection port are provided by an input waveguide rod and output waveguide rod.

7. The system of claim 1, wherein the tunable laser spectrometer sweeps its wavelength in a spectral band including 2.3 micrometers and/or 6.5 micrometers.

8. The system of claim 1, wherein the tunable laser spectrometer sweeps through greater than 100 nanometers.

9. The system of claim 1, wherein the tunable laser spectrometer sweeps its wavelength from about 2.2 to 2.4 micrometers in wavelength.

10. The system of claim 1, wherein the monitoring system includes a probe that is inserted into a bioreactor.

11. The system of claim 1, wherein the monitoring system includes a sample interface for receiving drops of a sample extracted from a bioreactor.

12. The system of claim 1, further comprising a controller that monitors a response of the sample photodetector and the reference photodetector to resolve an absorption spectra of a sample in the sample detection region.

13. The system of claim 12, wherein the controller compensates for noise associated with ripple from the optical fiber.

14. The system of claim 12, wherein the controller compensates based on a temperatures detected by one or more thermistors associated with the reference photodetector and the sample photodetector.

15. The system of claim 1, wherein no optical fiber is provided after the reference detector to the sample photodetector.

16. A monitoring system comprising:
a tunable laser spectrometer for generating a swept wavelength signal;
a reference photodetector for detecting the swept wavelength signal from the tunable laser spectrometer;
a polarizer for improving a polarization of the swept wavelength signal prior to the reference photodetector; and
a sample photodetector for detecting the swept wavelength signal after transmission through a sample detection region.

17. A probe for a bioreactor, comprising:
ports defining a sample detection region in the bioreactor;
a reference photodetector for detecting an optical signal received from a polarizer and prior to the sample detection region; and
a sample photodetector for detecting the optical signal after transmission through the sample detection region.

18. A method for on-line or off-line monitoring of a bioreactor, the method comprising:
generating a swept wavelength signal;
transmitting the swept wavelength signal to a sample detection region for a sample of the bioreactor;
polarizing the swept wavelength signal;
detecting the swept wavelength signal after polarization and prior to transmission through the sample detection region;
detecting the swept wavelength signal after transmission through the sample detection region; and
resolving an absorption spectra of a sample in the sample detection region with reference to the swept wavelength signal before and after transmission through the sample detection region.
